(19) United States
(12) Patent Application Publication
(10) Pub. No.: US 2025/0099101 A1
(43) Pub. Date: Mar. 27, 2025

(54) Devices and methods for percutaneous bypass

(71) Applicants: Sorin Grunwald, Pantin (FR); Alexandros Mallios, Paris (FR)

(72) Inventors: Sorin Grunwald, Pantin (FR); Alexandros Mallios, Paris (FR)

(21) Appl. No.: 18/727,208

(22) PCT Filed: Jan. 7, 2023
(86) PCT No.: PCT/EP2023/050250

§ 371 Date: Jul. 8, 2024

Related U.S. Application Data
(60) Provisional application No. 63/298,108, filed on Jan. 10, 2022.

(51) Int. Cl. A61B 17/11; A61B 17/00; A61B 17/12

(52) U.S. Cl. CPC A61B 17/11; A61B 17/12031; A61B 17/12109; A61B 17/12172; A61B 2017/00557; A61B 2017/00862; A61B 2017/00867; A61B 2017/1107; A61B 2017/1135

(57) ABSTRACT

The present invention relates generally to devices for percutaneous, transcatheter anastomosis and bypass graft creation without the necessity to employ sutures. Such bypass grafts can be used for bypass surgery targeting arteries and/or veins or for vascular access for hemodialysis. In one embodiment of the present invention, a device is disclosed herein that consists of a stent graft with inflatable seals (200). The seals that are attached to the stent graft are inflated inside and outside the target blood vessel to seal the anastomosis location. These inflatable seals also act as a fixation mechanism of the stent graft to the blood vessel wall. According to the present invention, a bypass is achieved (100) by percutaneously deploying a first device to a first target blood vessel and a second device to a second target blood vessel and by connecting the two devices using a conduit-to-conduit connector disclosed herein (400).

### REFERENCES CITED

### Cross Reference to Related Applications

This application claims priority to U.S. Provisional Patent Application Ser. No. U.S. 63/298,108 filed on Jan. 10, 2022 (Devices and methods for percutaneous bypass).

### U.S. PATENT DOCUMENTS

| | | | |
|---|---|---|---|
| 5,755,775 | May 1998 | Trerotola et al. | Percutaneous stent-graft and method for delivery |
| | | | thereof |
| 5,755,778 | May 1998 | Kleshinski | Anastomosis device |
| 5,769,882 | June 1998 | Fogarty et al. | Methods an apparatus for conformably sealing |
| | | | prostheses within body lumens |
| 5,797,920 | August 1998 | Kim | Catheter apparatus and method using a shape- |
| | | | memory alloy cuff for creating a bypass graft in-vivo |
| 5,848,964 | December 1998 | Samuels | Temporary inflatable filter device and method of |
| | | | use |
| 5,941,908 | August 1999 | Goldsteen et al. | Artificial medical graft with a releasable retainer |
| 6,015,431 | January 2000 | Thornton et al. | Endoluminal stent-graft with leak-resistant seal |
| 6,036,702 | March 2000 | Bachinski et al. | Medical grafting connectors and fasteners |
| 6,402,764 | June 2002 | Henricksen et al. | Everter and threadthrough system for attaching |
| | | | graft vessel to anastomosis device |
| 6,648,901 | November 2003 | Fleischman et al. | Anastomosis system |
| 6,776,785 | August 2004 | Yencho et al. | Implantable superelastic anastomosis device |
| 7,762,977 | July 2010 | Porter et al. | Device and methods for vascular access |
| 8,277,465 | October 2012 | Beane et al. | Apparatus and method for connecting a conduit to |
| | | | a hollow vessel |
| 8,747,344 | June 2014 | Khan | Hybrid arteriovenous shunt |
| 8,900,288 | December 2014 | Chobotov et al. | Advanced endovascular graft delivery system and |
| | | | method of treatment |
| 9,055,946 | June 2015 | Yevzlin et al. | Anastomotic connector |
| 9,247,930 | February 2016 | Coleman et al. | Device and methods for occluding or promoting |
| | | | fluid flow |
| 9,597,443 | March 2017 | Yevzlin et al. | Anastomotic connector |
| 9,603,708 | March 2017 | Robin et al. | Low crossing profile delivery catheter for |
| | | | cardiovascular prosthetic implant |
| 9,826,695 | November 2017 | Stokes et al. | Devices for sealing a body lumen |
| 10,470,870 | November 2019 | Schreck et al. | Modular stent graft systems and methods with |
| | | | inflatable fill structures |
| 10,751,056 | August 2020 | Porter | Methods and apparatus for percutaneous bypass |
| | | | graft |
| 10,853,366 | November 2020 | Donadio, III et al. | Arterial and venous anchor devices forming an |
| | | | anastomotic connector and system for delivery |

### Other Publications

1. Agarwal A. K. et al., Innovations in vascular access for hemodialysis, Kidney International (2019) 95, 1053-1063
2. Allon M., Vascular Access for Hemodialysis Patients—New Data Should Guide Decision Making, American Society of Nephrology, CJASN Vol 14 Jun. 2019
3. Canaud B. et al., Vascular Access Management for Haemodialysis: A Value-Based Approach from NephroCare Experience, IntechOpen 2019
4. Ilie V. et al., Sutureless Microvascular Anastomosis: Literature Review, International Microsurgery Journal, 2019; 3(2):1
5. Mallios A., et al., Extra-anatomical endo-bypass between left arm vascular access and superior vena cava, Journal of Vascular Surgery, in print.
6. Mallios A. et al., Early results of percutaneous arteriovenous fistula creation with the Ellipsys Vascular Access System, Journal of Vascular Surgery, 2018
7. Al-Jaishi A. A. et al., Complications of the Arteriovenous Fistula: A Systematic Review, J Am Soc Nephrol 28:1839-1850, 2017

### Technical Field

The present invention relates generally to devices and methods that can be used for bypass procedures. More specifically, the present invention relates to percutaneous, transcatheter anastomosis and bypass graft creation without the necessity to employ sutures. Bypass grafts created according to the present invention can be used, amongst other applications, for peripheral vascular bypass surgery targeting arteries and/or veins and for vascular access for hemodialysis including arteriovenous grafts.

### Background

### Clinical Background

Worldwide, hemodialysis (HD) remains the prevalent dialysis modality for more than 2 million patients with end-stage renal disease. The health care expenditure to treat end-stage renal disease has increased to approximately $34 billion in 2015 in the United States alone. A significant portion of this expense is related to the establishment and maintenance of vascular access (VA) for HD.

Arteriovenous grafts (AVG) represent an important vascular access option for hemodialysis. Compared to the arteriovenous fistulas, the AVG has better mechanical strength, earlier use, and lower primary failure rates. Central venous catheters (CVC) are also used in specific cases for starting hemodialysis because they provide immediate vascular access. Compared to CVCs, the use of AVG results in lower risk of bloodstream infections, in a lower risk of central vein stenosis, and increased survival rates.

Worldwide, peripheral vascular disease (PVD) affects more than 200 million patients worldwide and is increasingly recognized as an important cause of cardiovascular mortality although it has been historically underappreciated compared with coronary artery disease and stroke.

Vascular bypass graft surgery represent an important option for treating advanced PVD. In the case of peripheral artery disease, minimally invasive treatment options such as angioplasty, stenting or atherectomy can be used. However, if the vascular blockage is in advanced stage, in a difficult to access location, or is too hardened to be removed effectively, bypass graft surgery is often the only treatment available to avoid severe complications, such as amputation or even death.

### Clinical Need

Most of the innovative efforts for improving bypass surgery have been focused on coronary artery bypass grafts (CABG). What is needed are devices and methods for minimally invasive procedure to create and maintain arteriovenous grafts for hemodialysis access. What is further needed are devices and methods for minimally invasive procedures to create a vascular bypass between an unobstructed peripheral artery or vein and an obstructed peripheral artery or vein. Such devices should reduce the risks associated with establishing and maintaining vascular access, decrease time to first use, increase long-term patency, minimize complications, simplify the clinical procedure, and reduce the burden on patients and on the healthcare system

### Related Art

U.S. Pat. No. 5,755,775 describes a stent-graft assembly for restoring blood flow between blood vessels. The stent-graft has a body implantable device and first and second retaining elements. It can be deployed percutaneously.

U.S. Pat. No. 5,755,778 describes an anastomosis device for use in forming a graft between two body members which includes a body member of fluid impervious vessel compatible material.

The outer surface of the body member carries a bio-adhesive which bonds the body member to the luminal walls of two vessels to be joined when the body member is expanded into contact with the vessel luminal walls.

U.S. Pat. No. 5,769,882 describes a tubular prosthesis implanted at a target location within a body lumen by embedding an expansible prosthesis body within a sealing layer. The sealing layer occludes at least a circumferential band within an interface region between the prosthesis body and the inner wall of the body lumen, thus providing for blockage of body lumen flow past prosthesis. The sealing layer may be introduced prior to or simultaneously with the prosthesis body.

U.S. Pat. No. 5,797,920 provides a catheter apparatus, an introducer system, and a methodology for creating a bypass on-demand between an unobstructed blood vessel such as the aorta and an obstructed blood vessel such as an obstructed coronary artery in-vivo using a shaped memory alloy cuff and a graft segment in tandem as a shunt. The deformable thermoelastic cuff is a substantially cylindrical-shaped collar which is open at each of its ends. The cuff is hollow; is substantially round or oval (in cross-sectional view); and has a determinable first configuration and dimensions initially which are deformed at will into a second memory-shaped configuration when placed at a temperature greater than about 25-35 degrees Celsius. The temperature-deformable cuff is intended to be positioned within the internal lumen of a blood vessel and become thermally deformed in-situ such that a portion of the cuff wall becomes positioned and secured to the internal lumen of the blood vessel. On the outer side of the blood vessel, the placement of a biocompatible adhesive at the puncture and graft juncture site places the bypass conduit in a fluid-tight setting.

U.S. Pat. No. 5,848,964 describes a device that features a catheter with an inflatable cuff at one end. During the insertion, the cuff is deflated and is wrapped about the catheter. The catheter features a center lumen and an inflation lumen that runs parallel to the central lumen and is used for the inflation and the deflation of the inflatable cuff. The distal port of the inflation lumen is in fluid communication with the inflatable cuff while the proximal port is connected to a means for inflating the cuff with inflation material. The inflatable cuff has a generally hollow cylindrical configuration with a relatively large central aperture. The cuff is permanently attached to the distal end of said catheter.

U.S. Pat. No. 5,941,908 describes a tubular artificial graft connector for attachment to a patient's tubular body tissue. The connector has an initially radially relatively small connector structure adjacent each of its ends. The initial relatively small size of each connector end facilitates insertion of that portion or the graft into the body tissue to which that connector structure is to make a connection. After a connector end is properly positioned relative to the body tissue, the connector end structure is radially enlarged to connect the graft to the body tissue.

U.S. Pat. No. 6,015,431 describes an implantable medical device that has a tubular member and a sealing member secured to the outer surface of the tubular member. The tubular member is expandable to engage an endolumenal wall. The seal member occludes flow around the tubular member between the outer surface of the member and the endolumenal wall. The tubular member may be a stent-graft. The seal member may be a flange that is oriented on the outer surface at one end of the stent-graft as a one-way valve against the blood flow.

U.S. Pat. No. 6,036,702 describes a body tissue graft that includes a frame structure made of a first elastic material, a covering of a second elastic material on the frame structure, the covering substantially filing the openings in the frame structure, and a connector connected to the frame structure. Special elements are secured to the connector structure that facilitate attachment to body tissue by circumferentially expanding in the body tissue. This may be done by using an inflatable balloon to expand the special elements.

U.S. Pat. No. 6,402,764 describes a device useful for attaching a graft vessel to an anastomosis device which can be used to attach a graft vessel to a target vessel without the use of conventional sutures.

U.S. Pat. No. 6,648,901 describes and end-side anastomosis system including a base for attachment to a graft, said base be configured to form a seal with an opening in a host vessel wall.

U.S. Pat. No. 6,776,785 describes a one-piece anastomosis device formed of a superelastic or pseudoelastic material which self-deforms or self-deploys from an insertion configuration to a tissue holding configuration. The self-deploying anastomosis device does not rely on a temperature transformation to achieve deployment.

U.S. Pat. No. 7,762,977 contemplates a catheter section adapted for insertion into a vein and a graft section adapted for attachment to an artery. The arteriovenous graft comprises a connector for facilitating attachment of the catheter portion to the graft portion. The connector may be pre-connected to the graft portion or may be integral with the graft portion. The connector may have a securing system for resisting disconnection of joined components.

U.S. Pat. No. 8,277,465 provides a system and method for forming a side branch on a hollow vessel. The system comprises a positioning element adapted to hold the position of the side branch relative to the vessel. The positioning element may be an expansion element, which may be expandable from an unexpanded state. The expansion element may be a balloon, which may be in the shape of a circular toroid and may include a tension member that restricts the dimensions of the balloon. If the expansion element is a balloon, the means for expanding may comprise a syringe in fluid communication with the balloon.

U.S. Pat. No. 8,747,344 describes a surgically created hybrid arteriovenous shunt which comprises a flexible graft and a venous outflow catheter connected to the graft via surgical anastomosis over a cuff. The cuff comprises an inlet and an outlet, wherein the inlet is connected to the intake end of the subcutaneous graft and the outlet is connected to the intake of the venous catheter. In a preferred embodiment, the inside diameter of the cuff is graded to compensate for the size difference between the graft and the venous catheter. The cuff is preferably Teflon or Dacron and provides a secure fit for the arterial graft first diameter and the venous catheter second diameter.

U.S. Pat. No. 8,900,288 describes an endovascular graft made from an inflatable graft body section with one or more inflatable cuffs disposed at either end of the graft body section. At least one inflatable channel is disposed between and in fluid communication with the inflatable cuffs.

U.S. Pat. No. 9,055,946 describes an anastomotic connector comprising a generally tubular access port having a first end and a second end and a main body portion in fluid communication with the second end of the access port that is structured to be deployed within a fluid passageway. The main body portion includes an expandable mesh frame defining a pair of flanges extending outwardly from the second end of the access port and a retention strap extending across the second end of the access port. The pair of flanges and the retention strap are structured to exert a radial force on an internal surface of a fluid passageway when the mesh frame of the main body portion is expanded within the fluid passageway. Furthermore, the pair of flanges and the retention strap allow the passage of fluid to the distal tissues that the native fluid passage is supplying.

U.S. Pat. No. 9,247,930 describes devices and sutureless percutaneous methods for occluding or promoting fluid flow through openings. In an exemplary embodiment an occlusion device is provided having an outer elongated tubular body that is configured to expand and form proximal and distal wings proximate to opposed ends of the opening. The device can include a component to occlude flow through the tubular body and thus through the opening. When used for puncture closure, the device is radiopaque and visible under fluoroscopic control during all phases of deployment and implantation. The device consists of a delivery system with a premounted implant on its tip. Once located, the device is activated and delivers a small biocompatible stainless steel implant into the vascular wall thereby closing the arteriotomy and effecting hemostasis.

U.S. Pat. No. 9,597,443 describes an anastomotic connector comprising a tubular access port and an anchor with a plurality of fingers to be extended in a blood vessel connected to the tubular access port.

U.S. Pat. No. 9,603,708 describes a delivery catheter for deploying a cardiovascular prosthetic implant, wherein the implant comprises an inflatable cuff and a tissue valve coupled to the inflatable cuff. The inflatable cuff is inflated fully with a hardenable inflation media. The cuff comprises a thin flexible tubular material such as a flexible fabric or thin membrane with little dimensional integrity. Uninflated, the cuff is incapable of providing support. In one embodiment the cuff comprises Dacron, PTFE, ePTFE, TFE or polyester fabric as seen in conventional devices. The inflatable structure is formed by one or more inflation channels formed by a pair of distinct balloon rings or toroids and struts. The inflation channels have connection ports four coupling to the delivery catheter via position and fill lumen tubing. Inflation media, air or liquid can be introduced into the inflation channels through the connection ports.

U.S. Pat. No. 9,826,965 describes a sealing cuff configured to be positioned around a body lumen and a sealant. In one embodiment, the sealing cuff can form an enclosed loop around the anastomosis and can define an interior chamber for receiving sealant therein. The sealant cuff can ensure that the sealant remains in contact with the body lumen as the sealant cures and reinforces the anastomosis. Methods for sealing an anastomosis are also provided and include delivering sealing to the sealing cuff, inserting inflatable members so they positioned adjacent to the anastomosis, and delivering fluid to at least one of the inflatable members to expand an inner wall of the tubular organ.

U.S. Pat. No. 10,470,870 describes an apparatus including a stent graft that is at least partially insertable into a blood vessel. The apparatus further includes an inflatable fill structure fixed to a portion of the outside surface of the stent graft. The inflatable fill structure includes an outer membrane that is configured to extend beyond the end of the stent graft when the inflatable fill structure is in a filled state.

U.S. Pat. No. 10,751,056 describes methods and apparatus for a percutaneous bypass graft system including a graft section comprising a dual-side fixation system. The dual-sided fixation system may comprise a plurality of barbs configured to secure the graft section to an internal and external portion of a target vessel. A procedural sheath comprises a cuff at a distal end that is configured to be positions against an outer surface of the target vessel during the percutaneous procedure.

U.S. Pat. No. 10,835,366 describes arterial and venous anchor devices coupled by graft material to form an anastomotic connector. The arterial anchor device comprises a tubular main body including a distal end, the distal end defining a plurality of flanges being movable from a first loaded position to a second expanded position. The venous anchor device includes a tubular main body having a metal frame structure, the distal end including a plurality of barbs.

### SUMMARY

The present invention relates generally to devices and methods that can be used for bypass procedures. More specifically, the present invention relates to percutaneous, transcatheter anastomosis and bypass graft creation without the necessity to employ sutures. Bypass grafts created according to the present invention can be used, amongst other applications, for peripheral vascular bypass surgery targeting arteries and/or veins and for vascular access for hemodialysis including arteriovenous grafts. In one embodiment of the present invention, an anastomosis device disclosed herein consists of a stent graft with inflatable seals (200). The seals that are attached to the stent graft are inflated inside and outside the target blood vessel to seal the anastomosis location. These inflatable seals also act as a fixation mechanism of the stent graft to the blood vessel wall. According to the present invention, a bypass is achieved (100) by percutaneously deploying a first said anastomosis device to a first target blood vessel and a second said anastomosis device to a second target blood vessel and by connecting the two devices using a conduit-to-conduit connector disclosed herein (400). By using the devices and the methods disclosed herein one-to-many and many-to-one bypass paths can be also achieved.

### BRIEF DESCRIPTION OF THE DRAWINGS

FIG. 1 illustrates devices and methods for percutaneous bypass according to the present invention.

FIG. 2 illustrates an embodiment of an anastomosis device with inflatable seals for percutaneous delivery according to the present invention.

FIG. 3A illustrates an embodiment of percutaneous deployment of an anastomosis device in a deflated (uninflated) state according to the present invention.

FIG. 3B illustrates an embodiment of a percutaneously deployed and inflated anastomosis device according to the present invention.

FIG. 4A illustrates an embodiment of an end-to-end conduit-to-conduit connector for conduits of different sizes according to the present invention.

FIG. 4B illustrates an embodiment of a conduit connecting piece with fixation mechanism according to the present invention.

FIG. 4C illustrates an embodiment of an end-to-side conduit-to-conduit connector according to the present invention.

FIG. 5 illustrates an embodiment of a 1-to-2 Y-shaped conduit-to-conduit connector according to the present invention.

FIG. 6 illustrates an embodiment of inflatable device seals according to the present invention.

FIG. 7 illustrates another embodiment of inflatable device seals according to the present invention.

### DETAILED DESCRIPTION

While the invention is described in detail with reference to the preferred embodiment thereof, it will be apparent to one skilled in the art that various changes and modifications can be made and equivalents employed, without departing from the present invention. In order to streamline the disclosure of the present invention, not all elements of one embodiment are described in detail but only those specifically targeted in that particular embodiment. For example, an embodiment may be illustrated comprising a hemostatic valve or without such valve. It will be apparent to one skilled in the art that such elements, like the above-mentioned valve, may be or not be present in all configurations and drawings without affecting the objective, spirit, and scope of the present invention. In addition, modifications may be made to adapt a particular situation, material, shape, dimension, processor method to the objective, spirit, and scope of the present invention. All such modifications are intended to be within the scope of the claims appended hereto. In the present disclosure, like-numbered components of embodiments generally have similar features, and thus within a particular embodiment each feature of like-numbered component is not necessarily fully elaborated upon in the description of the particular embodiment.

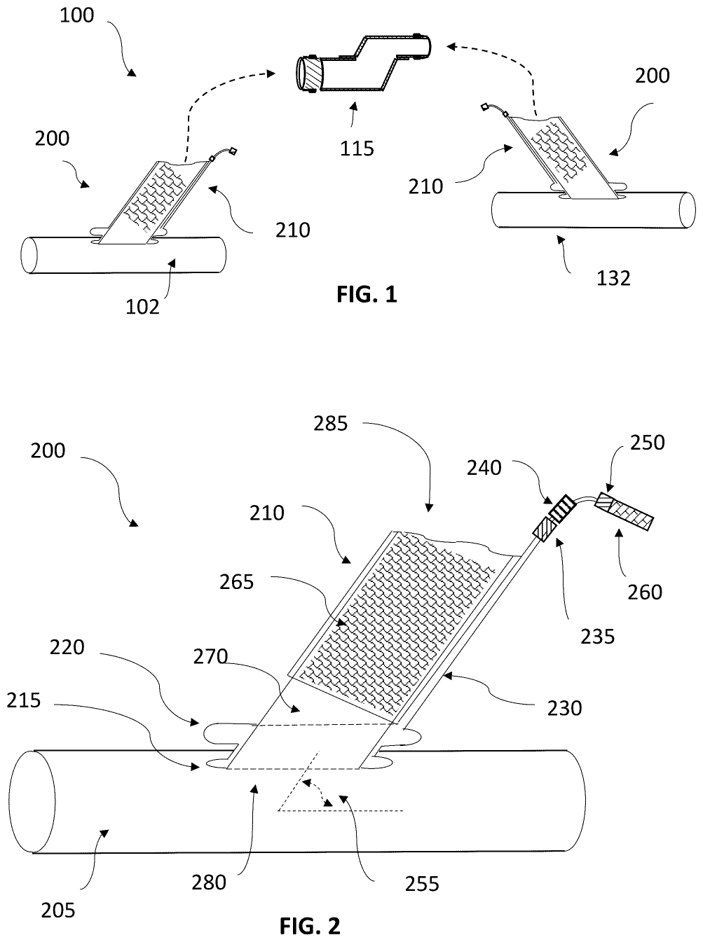

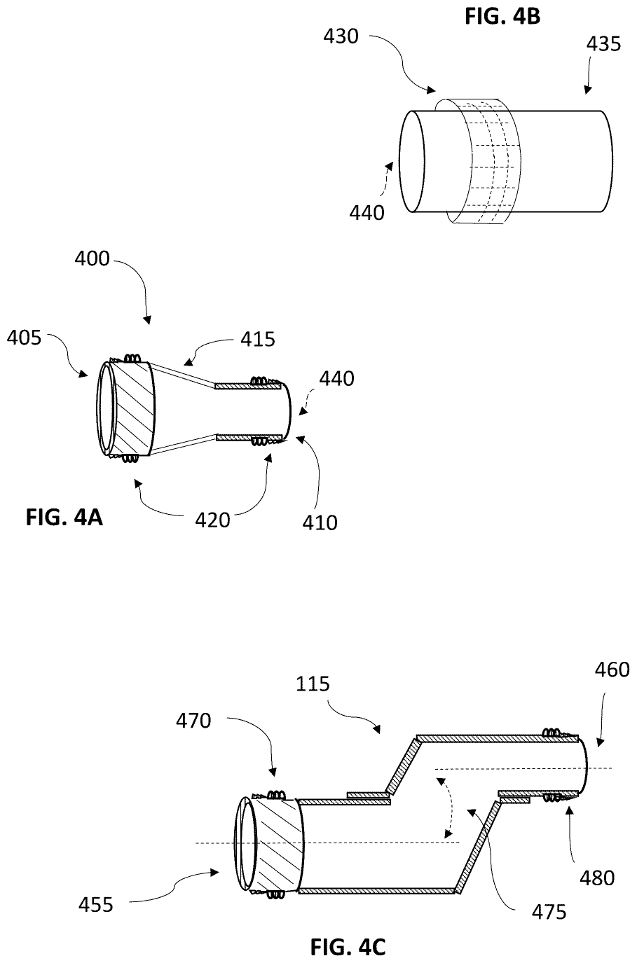

FIG. 1 illustrates devices and methods for percutaneous bypass according to the present invention. In one embodiment of the present invention, a first anastomosis device 200 (further described herein in FIG. 2) is inserted in a blood vessel 102 and can be connected to one end of a conduit-to-conduit connector 115 (further described herein in FIG. 4). A second anastomosis device 200 can be connected at the second end of the conduit-to-conduit connector 115 and attached to a second blood vessel 132, thus creating a bypass 100 between blood vessels 102 and 132. Blood vessels 102 and 132 can be arteries and/or veins as required by the medical bypass procedure.

According to the present invention, in one embodiment, a method of creating a percutaneous bypass comprises the following steps:
1. Gain percutaneous access to a first blood vessel 102.
2. Percutaneously deploy first anastomosis device 200.
3. Gain percutaneous access to a second blood vessel 132.
4. Percutaneously deploy second anastomosis device 200.
5. Tunnel the conduit 210 of first anastomosis device 200 and the conduit 210 of second anastomosis device 200 towards an accessible connection location.
6. At the connection location, connect conduit 210 of first anastomosis device 200 and the conduit 210 of second anastomosis device 200 using connector 115 as described further herein, thus establishing the bypass conduit between blood vessels 102 and 132.

Similarly, but not restrictively, in another embodiment of the present invention, a bypass connection can be realized using device 200 anastomosed to a first blood vessel and conduit-to-conduit connector 115 connected at its second end to a graft, stent-graft or catheter.

In yet another embodiment of a bypass method according to the present invention, the bypass between a first blood vessel 102 and a second blood vessel 132 can be achieved without the use of connector 115. The conduit 210, e.g., a stent-graft, of an anastomosis device 200 attached to a first blood vessel 102 can be directly sutured to a second blood vessel 132.

FIG. 2 illustrates an embodiment of an anastomosis device 200 for percutaneous transcatheter delivery configured to suturelessly anastomose a conduit to a blood vessel according to the present invention. On FIG. 2, device 200 is attached to a blood vessel 205. In one embodiment of the present invention, the device 200 consists of a conduit 210 with two ends: a distal end 280 that connects to a blood vessel and a proximal end 285. In one embodiment of the present invention, at the distal end 280 of the device 200, two inflatable elements are attached: a distal inflatable element 215 and a proximal inflatable element 220. Elements 215 and 220 serve as seals and prevent blood from leaking out of the blood vessel. They also serve to anchor the conduit 210 to the blood vessel wall providing for a reliable anastomosis between conduit 210 and blood vessel 205.

In a preferred embodiment, inflatable elements 215 and 220 are ring-shaped around the conduit 210.

In some embodiments of the present invention, only distal inflatable element 215 is attached to or integrated in conduit 210.

In one embodiment of the present invention, the conduit 210 is a stent graft.

In another embodiment of the present invention, the conduit 210 is a catheter.

In another embodiment of the present invention, the conduit 210 consists of two or more segments, for example a graft segment 270 and a stent graft segment 265.

In some embodiments of the present invention, the elements 215 and 220 are an integral part of graft segment 270.

In other embodiments of the present invention, the elements 215 and 220 represent a double inflatable cuff attached to conduit 210, said conduit 210 being, for example, a catheter or a stent graft.

In more embodiments of the present invention, graft tube segment 270 is made of PTFE (polytetrafluoroethylene), ePTFE, Dacron, silicon, polyurethane, TFE or polyester fabric as seen in conventional medical devices such as prosthetic arteriovenous grafts. The fabric thickness and weave density may be adjusted to a very tight weave to prevent blood from leaking through the fabric.

In more embodiments of the present invention, stent-graft tube segment 265 is composed of fabric supported by a metal mesh called a stent as seen in conventional medical devices such as aortic stent-grafts and covered stents for peripheral vascular interventions, whereby the stent material can be, for example, nitinol or stainless steel, and the fabric material can be, for example, PTFE.

During the percutaneous deployment procedure, the conduit 210 is introduced in blood vessel 205 with the elements 215 and 220 uninflated (deflated). After proper positioning of the conduit 210 relative to the blood vessel wall, the inflatable elements 215 and 220 are inflated to expand radially and/or axially. The distal element 215 is inflated in the interior of the blood vessel. The proximal element 220 is inflated at the exterior of the blood vessel. Said inflatable elements are positioned such as to, when inflated, reliably attach to the blood vessel wall sandwiching the vessel wall between them and thus creating an anastomosis between the blood vessel 205 and the conduit 210. When inflated, the elements 215 and 220 serve as seals and do not allow the blood to flow outside the anastomosis between blood vessel 205 and conduit 210.

In one embodiment of the present invention, inflatable elements 215 and 220 are of different sizes and asymmetrical with respect to the longitudinal axis of the conduit 210. The angle 255 between the longitudinal axes of the blood vessel 205 and of the conduit 210, i.e., the angle of deployment of the inflatable elements 215 and 220, can be selected as a function of the anatomy and of the anastomosis location to optimize blood flow.

In one embodiment of the present invention, the device 200 further consists of an inflation/deflation conduit or fill tube or lumen 230 that allows the passage of the inflation material towards and from inflatable elements 215 and 220. The lumen or lumens 230 can be built inside or outside the lumen of conduit 210. The conduit 230 is connected to valve 235 that prevents the fil medium to exit inflatable elements 215 and 220 and conduit 230. Valve 235 is connected to detaching/separation mechanism 240. An inflation port 250 allows for connection to the inflation conduit 230 of an inflation device 260, e.g., a syringe or a pump. The inflatable elements 215 and 220 can be filled through the inflation port 250 with a fill medium, e.g., a gas or a liquid.

In some embodiments, the fil medium may be, for example, a liquid such as saline. In some embodiments, contrast medium may be added to the fill medium to visualize the inflatable elements under fluoroscopy. In other embodiments, the fill medium can be a hardenable and/or polymer fill medium.

In one embodiment of the present invention, the inflatable elements 215 and 220 are made of biocompatible polymers. These elements can be manufactured by blow molding extruded tubes made from polymers with sufficient flexibility for deflation and inflation, and good mechanical strength to resist burst pressure when expanded, e.g., high durometer polyether block amide (PEBA).

In one embodiment of the present invention, inflatable elements 215 and 220 are formed as balloon rings.

In another embodiment of the present invention, the inflatable elements 215 and 220 are formed as balloon rings and a connecting balloon toroid that penetrates the blood vessel wall.

Inflatable elements 215 and 220 can be secured to graft segment 270 in any of a variety of manners. In one embodiment of the present invention, elements 215 and 220 are secured within graft folds, whereby the folds, in turn, are secured by sutures or stiches to the graft.

At the proximal end of device 200, the inflation side-arm port 250 emerges from the conduit 210. Both the angle of departure and length of the side-arm port may be variable. Side-arm port 250 and conduit 210 may be molded/extruded as a single piece or, alternatively, the side-arm port may be bonded to the conduit.

In one embodiment of the present invention, the inflation lumen 230 features a valve 235. Side-arm port 250 allows passage of inflation material into the inflation lumen 230 via injection with a syringe and/or an inflation pump 260. While the syringe is attached to the side-arm port 250, valve 235 allows inflation material to flow either into or out of inflation lumen 230 so that the inflatable elements 215 and 220 can be inflated or deflated. When said inflatable elements are inflated and the syringe 260 is removed, valve 235 prevents the inflation material from escaping out of the inflation lumen 230 and of the inflatable elements 215 and 220. As a results, the inflatable elements are maintained in an inflated state.

In one embodiment of the present invention, valve 235 is a 3-way valve. Applying positive pressure (infusion) using syringe and/or inflation pump 260 opens valve 235 and allows for injecting inflation material into the inflatable elements 215 and/or 220. Applying negative pressure (aspiration) using syringe and/or inflation pump 260 opens valve 235 and allows for absorbing inflation material from the inflatable elements 215 and/or 220. When neither positive nor negative pressure is applied to valve 235, the valve is closed thus preventing inflating material from exiting inflatable elements 215 and/or 220.

In one embodiment of the present invention, inflatable elements 215 and 220 are connected and can be inflated via a single inflation lumen 230 and port 250. In another embodiment of the present invention, the inflatable elements 215 and 220 can be inflated/deflated separately via their own dedicated inflation lumens and inflation ports.

In one embodiment of the present invention, a detach mechanism 240 is used to separate the extracorporeal piece of the inflation/deflation lumen 230 from the intracorporeal piece of said lumen. In various embodiments of the present invention, the detach mechanism is represented by perforations, reduced wall thickness, etc., such that when pulling, pulling, and/or rotating the fill lumen 230 from outside the body, the material can break at the designed location and the extracorporeal piece of the lumen can separate from the intracorporeal piece.

In another embodiment of the present invention, mechanism 240 is used for both detaching and reattaching the extracorporeal piece of the inflation/deflation lumen 230 from and to the intracorporeal piece of said lumen. In various embodiments of the present invention, the detach/attach mechanism is represented by threaded ends, bayonet locks and alike.

In one embodiment of the present invention, inflatable elements 215 and/or 220 can be deflated at any time after deployment using inflation/deflation lumen 230 thus allowing for the anastomosis device 200 to be removed from the anastomosis site.

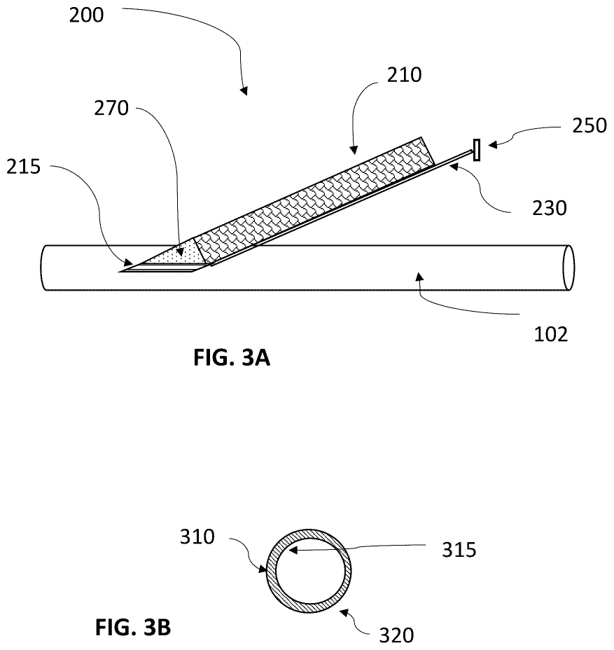

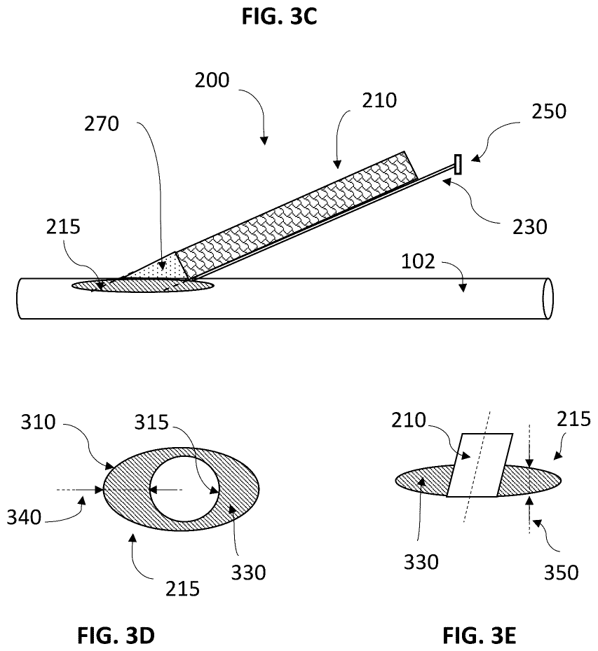

FIG. 3A illustrates the deployment in a blood vessel 102 of anastomosis device 200 configured with only a distal inflatable element 215 according to the present invention. During deployment of the device 200 in the blood vessel through a procedural sheath and/or over a guidewire, the inflatable element 215 is deflated (uninflated). After the device has been placed at the desired location in the blood vessel, the procedural sheath is retracted and the inflatable element 215 is inflated using the inflation lumen 230 and inflation port 250. By inflating element 215, the graft segment 270 of the device takes a compliant shape according to the anastomosis location. In one embodiment of the present invention, the stent-graft segment 265 is self-expandable and self-expands once the procedural sheath has been retracted.

FIG. 3B illustrates a cross-section of inflatable elements 215 and/or 220 in the deflated state during deployment and before inflation. The inner diameter 315 is determined by the size and shape of the conduit 210. The outer diameter 310 of inflatable elements 215 and/or 220 is determined by the profile 320 of said elements in the deflated state.

FIG. 3C illustrates device 200 deployed in a blood vessel 102 retracted against the vessel wall after element 215 has been inflated according to the present invention. The inflated element 215 acts as a seal preventing blood from leaking outside the blood vessel.

FIG. 3D illustrates a cross-section of inflatable elements 215 and/or 220 in the inflated state after deployment. The inner diameter 315 is determined by the size and shape of the conduit 210. The outer diameter 310 is determined, amongst other factors, by the size 340 of said element profile/shape 330.

FIG. 3E illustrates a transversal profile of inflatable elements 215 and/or 220 in a ring-shaped inflated state around conduit 210 after deployment and inflation. The element profile/shape 330 is determined, amongst other factors, by the height 350 of said element profile.

According to the present invention, a variable profile 330 of inflatable elements 215 and/or 220 in inflated state can be achieved in a number of inventive ways. In one embodiment of the present invention, the material of said inflatable elements has variable strength and rigidity across the surface, thus allowing for desired shapes of said elements in inflated state.

In another embodiment of the present invention, the material of inflatable elements 215 and/or 220 are reinforced in specific location in order to provide the desired shape in inflated state. In one embodiment of the present invention, specific material reinforcement is achieved by using struts and strut segments of superelastic materials, e.g., Nitinol integrated and distributed at desired locations within the material of said inflatable elements.

FIG. 4A illustrates an embodiment of a conduit-to-conduit connector 400 for conduits of different diameters 405 and 410. In one embodiment of the present invention, conical segment 415 of the connector ensures a smooth transition between the two differently sized conduits. Connector 400 can be provided in different sizes to accommodate different sizes of conduits. Connector 400 is made of any appropriate biocompatible material for subcutaneous placement, e.g., implantable formulations of polymers, stainless steel, titanium. Fixation mechanisms 420 are provided at both ends of the connector to allow for the attachment of conduits.

In one embodiment of the present invention, external rings can be used with the conduits for coupling with the fixation mechanism 420 as illustrated in FIG. 4B. In order to connect a conduit 435 to a connector 400 (440), a matched and appropriately sized ring 430 is placed over the conduit 435. Conduit 435 is then pushed over the fixation mechanism 410 or 405 according to the conduit and ring size. Pushing the ring 430 over the thus attached conduit and over the fixation mechanism locks the conduit in place over the connector 400.

In more embodiments of the present invention, fixation mechanisms 405 and 410 consists of threaded segments. In such embodiments, the ring 430 can be appropriately threaded to match the threads of segments 405 and 410. Ring 430 can be screwed over the conduit 435 and into the fixation threads 405 and 410.

In more embodiments of the present invention, the external rings are rotating rings made of biocompatible rigid material, plastics or metal or a combination thereof. In more embodiments of the present invention, ring 430 is made of biocompatible elastic material and simply pulled over the conduit 435 and the fixation mechanisms 405 and 410.

In one embodiment of the present invention, the conduit-to-conduit connector is an end-to-end connector 400. In another embodiment of the present invention, the conduit-to-conduit connector is an end-to-side connector 115 as illustrated in FIG. 4C. The end-to-side connector 115 is connected to conduit 435 similarly to the end-to-end connector 400. In order to connect conduit 435 to connector 115, a matched and appropriately sized ring 430 is placed over the conduit 435. Conduit 435 is then pushed over the fixation mechanism 470 or 480. Pushing the ring 430 over the thus attached conduit and over the fixation mechanism locks the conduit in place over the connector 115.

In more embodiments of the present invention, the angle 475 between the longitudinal axis corresponding to end 455 and the longitudinal axis corresponding to end 460 of connector 450 can have different values from parallel (0 degrees) to perpendicular (90 degrees) and to a U shape connector (180 degrees).

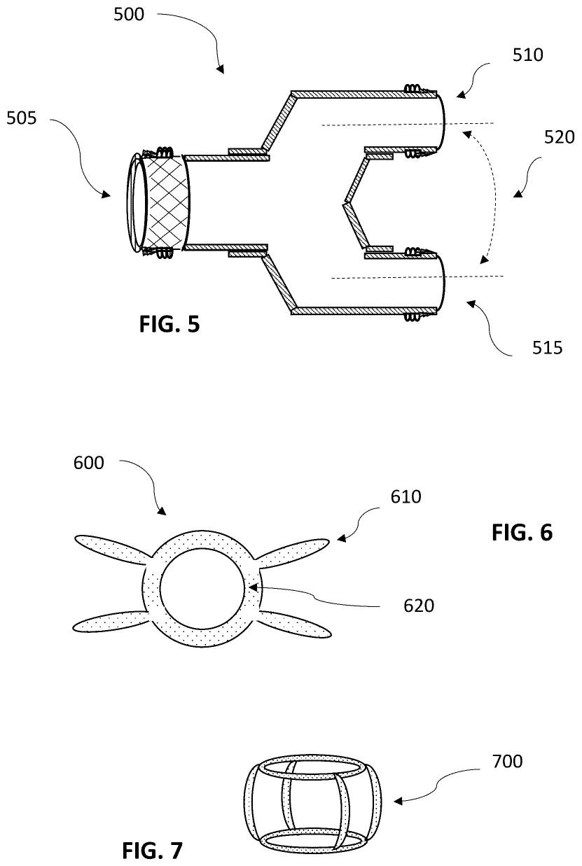

FIG. 5 illustrates an embodiment of a 1-to-2 (one-to-two and/or two-to-one) Y-shaped conduit-to-conduit connector 500 according to the present invention. In one embodiment of a bypass method according to the present invention, a bypass can be achieved from a first blood vessel 102 towards two other blood vessels using connector 500. In one embodiment of the present invention, an anastomosis device 200 attached to an artery 102 is connected to end 505 of connector 500. In another embodiment of the present invention, an anastomosis device 200 attached to a vein 102 is connected to the end 505 of connector 500. In one embodiment of the present invention, ends 510 and 515 of connector 500 can be connected to two veins using two anastomosis devices 200. In another embodiment of the present invention, ends 510 and 515 of connector 500 can be connected to two arteries using two anastomosis devices 200.

In more embodiments of the present invention, conduit-to-conduit connector 500 can be used to connect 1-to-2 and/or 2-to-1 any combination of arteries and veins.

In more embodiments of the present invention, connector 500 is a one-to-many or a many-to-one conduit-to-conduit connector.

Connector 500 is connected to conduits similarly to the end-to-end connector 400. In order to connect a conduit 435 to connector 500, a matched and appropriately sized ring 430 is placed over the conduit 435. Conduit 435 is then pushed over one fixation mechanism at one of the connector ends 505, 510, or 515. Pushing the ring 430 over the thus attached conduit and over the fixation mechanism locks the conduit in place over the connector 500.

In more embodiments of the present invention, the angle 520 between the longitudinal axes of corresponding ends 510 and 515 can have different values from parallel (0 degrees) to perpendicular to each other (90 degrees), to a T shape connector (180 degree) and to a W shape connector (360 degrees).

In more embodiments of the present invention, connector 500 is made of biocompatible thermoplastics or metal and metal alloys, or a combination thereof, for example TPU, PEEK, Titanium, stainless steel.

In another embodiment of the present invention, any of the inflatable elements 215 and 220 is formed by balloon segments 600 oriented like fingers 610 around a central ring 620 as illustrated in FIG. 6. In one embodiment of the present invention, said central ring is itself a balloon. In another embodiment of the present invention, said central ring is made of nitinol, stainless steel, or similar materials. In one embodiment of the present invention, fingers shape 600 is formed using reinforcement struts made of a supraelastic, self-expandable material such as nitinol whose finger shape is set using a shape-setting process. In one embodiment of the present invention, the reinforcement struts can be integrated in the balloon material during balloon fabrication in the balloon forming machine after balloon extrusion. In another embodiment of the present invention, the strut structure is covered by the balloon polymer membrane. In another embodiment of the present invention, finger shape 600 is formed by means of appropriate distribution of rigidity and elasticity properties of the balloon material, for example at the junction between the central ring and the extended fingers.

In another embodiment of the present invention, inflatable element 215 has a basket structure 700 as illustrated in FIG. 7, whereby the basket structure, after inflation, occupies the whole blood vessel cross section and blood continues to flow in between the balloon basket struts. In one embodiment of the present invention, basket shape 700 is formed using reinforcement struts made of a supraelastic, self-expandable material such as nitinol whose basket shape is set using a shape-setting process. In one embodiment of the present invention, the reinforcement struts can be integrated in the balloon material during balloon fabrication in the balloon forming machine after balloon extrusion. In another embodiment of the present invention, the strut structure is covered by the balloon polymer membrane. In another embodiment of the present invention, basket shape 700 is formed by means of appropriate distribution of rigidity and elasticity properties of the balloon material, e.g., at bend locations.

1. An anastomosis device (200) configured to suturelessly anastomose a conduit to a blood vessel, said device comprising:
an intracorporeal conduit (210) configured to provide a fluid path to and from a blood vessel or graft, wherein said intracorporeal conduit comprises a distal segment, a proximal segment, and at least one inflation/deflation lumen (230), said distal segment being fixedly attached to and within at least one inflatable element surrounding said intracorporeal conduit; and
at least one inflatable element (215) configured to be inflated inside a blood vessel or graft, suturelessly anastomose the distal end of said intracorporeal conduit to said blood vessel or graft, and seal the anastomosis site,
characterized in that the profile and shape of said inflatable element, when inflated, are customized by localized reinforcements made of supraelastic shape-memory material pre-shaped through a process of shape setting.

2. (canceled)

3. (canceled)

4. The anastomosis device according to claim 1, characterized in that the material reinforcements are configured such that said inflatable elements, when inflated, are at an angle (255) of 15 to 45 degrees with respect to the longitudinal axis of the blood vessel.

5. (canceled)

6. (canceled)

7. (canceled)

8. The anastomosis device according to claim 1, characterized in that at least one inflatable element is configured as fingers around a central ring (600).

9. The anastomosis device according to claim 1, characterized in that at least one inflatable element is configured as a basket (700).

10. (canceled)

11. The anastomosis device according to claim 1 further comprising a second inflatable element (220), characterized in that the first inflatable element inflated inside the blood vessel is smaller and thinner than the second inflatable element inflated outside the blood vessel in order for the first inflatable element to minimize impact on blood flow and for the second inflatable element to ensure increased sealing and reliable fixation at the anastomosis site.

12. (canceled)

13. (canceled)

14. (canceled)

15. A percutaneous bypass method (100), the method characterized in that it uses two anastomosis devices according to claim 1:
gaining percutaneous access to a first blood vessel at a first bypass target location;
percutaneously deploying a first anastomosis device according to claim 1 to the first target location;
gaining percutaneous access to a second blood vessel at a second bypass target location;
percutaneously deploying a second anastomosis device according to claim 1 to the second target location;
tunneling the intracorporeal conduit of first anastomosis device according to claim 1 and the intracorporeal conduit of second anastomosis device according to claim 1 towards an accessible connection location;
connecting the intracorporeal conduit of said first anastomosis device to the intracorporeal conduit of said second anastomosis device using the conduit-to-conduit connector, thus establishing the bypass conduit between the first and the second blood vessels.
